(19) United States
(12) Patent Application Publication
(10) Pub. No.: US 2022/0028534 A1
(43) Pub. Date: Jan. 27, 2022

(54) CLINICAL DECISION-MAKING SUPPORT APPARATUS, CLINICAL DECISION-MAKING SUPPORT SYSTEM, AND CLINICAL DECISION-MAKING SUPPORT METHOD

(71) Applicant: CANON MEDICAL SYSTEMS CORPORATION, Otawara-shi (JP)

(72) Inventors: Atsuko SUGIYAMA, Nasushiobara (JP); Junko SHIBATA, Nasushiobara (JP)

(73) Assignee: CANON MEDICAL SYSTEMS CORPORATION, Otawara-shi (JP)

(21) Appl. No.: 17/380,466

(22) Filed: Jul. 20, 2021

(30) Foreign Application Priority Data
Jul. 27, 2020 (JP) 2020-126777

(51) Int. Cl. G16H 40/20; G16H 50/20; G16H 80/00; G10L 15/22; G06F 40/40

(52) U.S. Cl. CPC G16H 40/20; G16H 50/20; G06F 40/40; G10L 15/22; G16H 80/00

(57) ABSTRACT

A clinical decision-making support apparatus according to an embodiment includes: processing circuitry that acquires disease, patient, medical, and personal information about at least an event that is specific to the subject, specifies an option about a treatment method for the disease, based on the disease, patient, and medical information, decides a treatment schedule about the method for the option, based on the disease, patient and medical information, and calculates an event schedule about the specific event in a period for which the method for the option is to be performed, based on the personal information, the method, and the treatment schedule; and a communication interface that transmits, to a terminal apparatus, display data expressing the event schedule and the event related to the treatment schedule, and the treatment schedule for each option in time series.

### CROSS-REFERENCE TO RELATED APPLICATIONS

This application is based upon and claims the benefit of priority from Japanese Patent Application No. 2020-126777, filed on Jul. 27, 2020; the entire contents of which are incorporated herein by reference.

### FIELD

Embodiments described herein relate generally to a clinical decision-making support apparatus, a clinical decision-making support system, and a clinical decision-making support method.

### BACKGROUND

Conventionally, shared decision making (hereinafter, SDM) has become popular in which a doctor and a patient share information, purposes, and responsibilities when deciding the patient's treatment method. However, when treatment involves various treatment methods, for example, pre-operative chemotherapy, surgery, radiation therapy, post-operative pharmacotherapy, and breast reconstruction in a case of treating breast cancer, the patient digests each treatment method presented by the doctor dispersedly at each stage of the treatment, comprehends them chronologically, and chooses the treatment method each time. It has been suggested that the patient can choose treatment methods with less regret and more satisfaction by considering not only the medical aspects of the treatment methods but also a variety of factors including money, family schedules, work, and whether having children in the future. However, for a patient who has been diagnosed with cancer, for example, it is not easy to look at these factors and make a fully satisfactory decision within a short period of time after diagnosis.

For example, breast reconstruction in a breast cancer patient is often performed after a total mastectomy and the following pharmacotherapy in accordance with the patient's condition. In this case, the breast cancer patient may decide whether to have the breast reconstruction after the pharmacotherapy is initiated. On the other hand, if the breast cancer patient chooses a method of inserting a tissue expander to expand the skin in the total mastectomy (immediate one-stage breast reconstruction, immediate two-stage breast reconstruction), the breast cancer patient needs to decide whether to have the reconstruction surgery before the total mastectomy. It is not easy for the breast cancer patient to understand the overall treatment process and the treatment methods and make such a decision considering the possible medical and personal events. In addition, if the patient wishes to have a baby after the treatment, a decision must be made whether to preserve eggs, sperms, or fertilized eggs before the chemotherapy is initiated. Here, it is not easy for the people concerned to understand when to make a decision, before the beginning of which treatment, on whether to see a reproductive medical doctor. Furthermore, since it is not easy to calculate the costs that will be incurred over time on the basis of the personal information along with the content and the process of the treatment, it is necessary to consult with specialists such as a cancer consultant, a social worker, the insurance department of workplace, and a person in charge of the patient's private insurance.

In addition, the choices involved in any treatment cannot all be determined before the treatment begins. For example, some treatment methods are determined based on the result of the specimen obtained in the previous surgery, or the effect of pre-operative pharmacotherapy may open up multiple surgical options. There are a number of milestones (determination branch points) for the doctor and the patient in the selection of chronological treatment methods. However, the current problem is that there is no way to know chronologically at which points in the treatment process decisions will need to be made before making such decisions in the treatment process. Typically, it is no more than just patient's own memorandum left after a decision is made and treatment is implemented.

### BRIEF DESCRIPTION OF THE DRAWINGS

FIG. 1 is a diagram illustrating a structure example of a clinical decision-making support system according to an embodiment;

FIG. 2 is a diagram illustrating one example of the clinical decision-making support system that is achieved on a cloud according to the embodiment;

FIG. 3 is a flowchart illustrating one example of the procedure of a decision-making support process according to the embodiment;

FIG. 4 is a diagram illustrating one example of a treatment schedule about an option A and an option B according to the embodiment;

FIG. 5 is a diagram illustrating one example of display content displayed on a display according to the embodiment;

FIG. 6 is a diagram illustrating one example of the display content displayed on the display according to the embodiment;

FIG. 7 is a diagram illustrating another example of the display content displayed on the display according to the embodiment; and

FIG. 8 is a diagram illustrating another example of the display content displayed on the display according to a second modification of the embodiment.

### DETAILED DESCRIPTION

A clinical decision-making support apparatus, a clinical decision-making support system, and a clinical decision-making support computer program are hereinafter described in detail with reference to the drawings. In the embodiment below, the components denoted with the same reference symbol perform the similar operation, and the redundant description is omitted as appropriate.

The clinical decision-making support apparatus described in the embodiment below includes processing circuitry and a communication interface. The processing circuitry acquires disease information about a disease of a subject, patient information of the subject, medical information about the disease, and personal information about at least a specific event of the subject. The processing circuitry specifies an option about a treatment method for the disease, based on the disease information, the patient information, and the medical information. The processing circuitry decides a treatment schedule about the treatment method for the option, based on the disease information, the patient information, and the medical information. The processing circuitry calculates an event schedule about the specific event in a period for which the treatment method for the option is to be performed, based on the personal information, the treatment method, and the treatment schedule. The communication interface transmits, to a terminal apparatus, display data expressing the event schedule and the event related to the treatment schedule and the treatment schedule for each option in time series.

### Embodiment

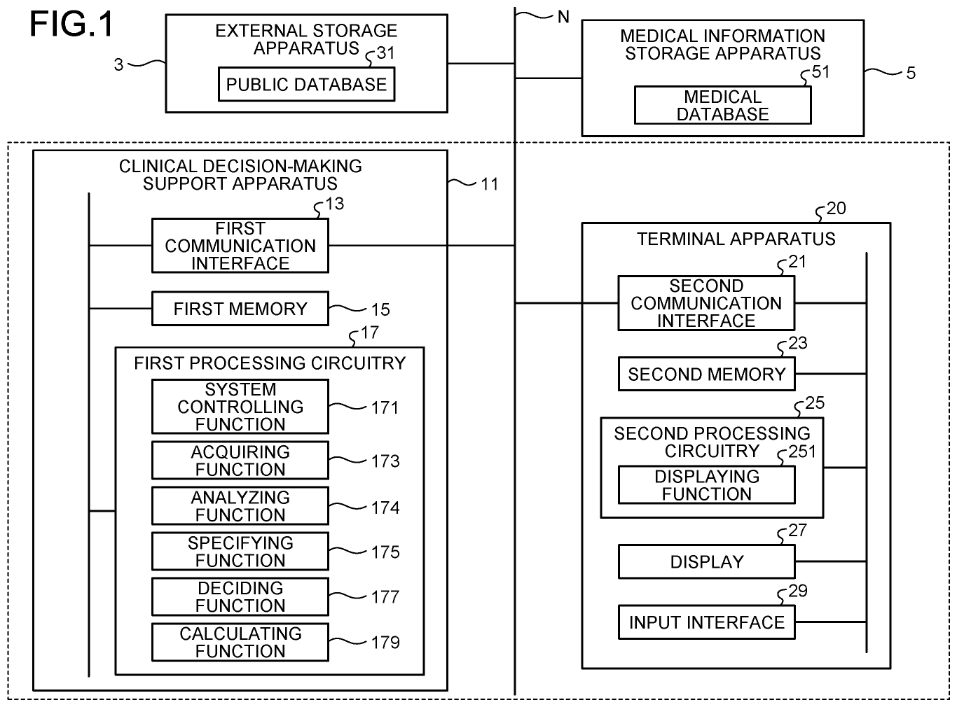

An entire structure of the clinical decision-making support system according to the embodiment is described. FIG. 1 is a diagram illustrating a structure example of a clinical decision-making support system 1 according to the embodiment. The clinical decision-making support system 1 includes a clinical decision-making support apparatus 11 and a terminal apparatus 20. The clinical decision-making support apparatus 11 and the terminal apparatus 20 are connected to each other through a network N, for example. The network N can be achieved in any mode as long as the clinical decision-making support apparatus 11 and the terminal apparatus 20 can communicate with each other either with or without a wire. To the network N, for example, an external storage apparatus 3 and a medical information storage apparatus 5 are connected either with or without a wire.

The external storage apparatus 3 stores a database 31 that is made public (hereinafter referred to as public database). The external storage apparatus 3 is installed outside the hospital, for example. The public database 31 stores therein, disease information about the disease, insurance information about the insurance coverage for the treatment of the disease, the medical information about the disease, and the like. Examples of the disease information include information about the guideline for the treatment methods of the disease (hereinafter referred to as disease guideline). Examples of the insurance information include information on insurance coverage, insurance benefits, and municipal benefits in general health insurance such as the national health insurance and private insurance by various insurance companies (including special contract for advanced medical care). Examples of the medical information include new papers and research reports about the disease.

The medical information storage apparatus 5 stores a database 51 about medical information (hereinafter referred to as medical database) therein. The medical information storage apparatus 5 is incorporated in a hospital information system (hereinafter referred to as HIS). The medical database 51 stores therein, for example, the patient information of the subject, the personal information about at least the event specific to the subject (that is, event unique to the subject), the insurance point according to the treatment method for the disease (hereinafter referred to as treatment insurance point), medical fee, standard monthly remuneration, the result of natural language processing (hereinafter referred to as language processing result) of the conversation between the subject and the doctor, and the like. The patient information includes the information in the electronic health record about the subject (for example, patient ID, name, birthday, disease name, weight, height, etc.).

The personal information includes, for example, the schedule information (action schedule) of the subject and the subject's family, the monthly remuneration (economic information) of the subject or the subject's household, the private insurance that the subject has and the content of that private insurance, the information about the health care held in the terminal apparatus 20, for example the subject's mobile terminal or wearable terminal, the items expressing the priority matters that the subject wants about the treatment of the subject's disease (hereinafter referred to as priority item), the language processing result, and the like. One example of the health care information is basal body temperature information. The object of the natural language processing may be audio data based on the conversation between the subject and the doctor or the chatbot and additionally, text data based on various search results by the subject. When the object of the natural language processing is the audio data, the language processing result corresponds to the result of analyzing the voice. The audio data and the text data are input to the terminal apparatus 20, for example. The language processing result is stored in the medical database 51 in a frequently asked question (FAQ) format, for example.

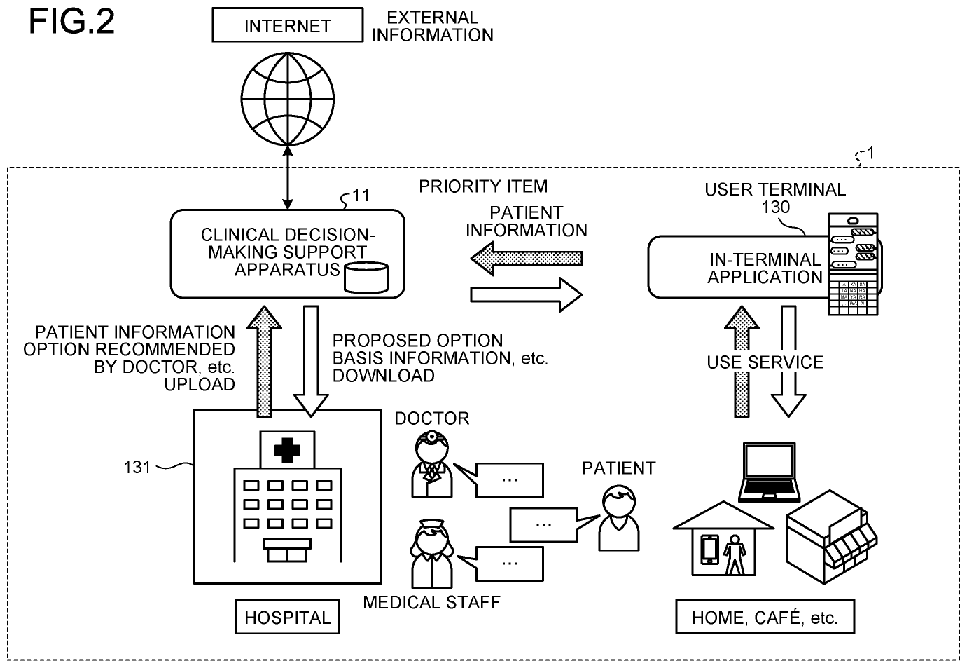

The clinical decision-making support apparatus 11 is achieved by a server apparatus, for example. Here, the terminal apparatus 20 is achieved as a client apparatus. Although one terminal apparatus 20 is illustrated in FIG. 1, a plurality of terminal apparatuses may be connected to the network N. Alternatively, the clinical decision-making support apparatus 11 may be incorporated in HIS or the like. Note that the client apparatus may be a user terminal 130 illustrated in FIG. 2.

The clinical decision-making support apparatus 11 includes a first communication interface 13, a first memory 15, and first processing circuitry 17. Note that at least one of server apparatuses on the network N and in the hospital may include at least one of a plurality of functions in the first processing circuitry 17, and at least one of the first memory 15 and the first communication interface 13.

Note that a computer (server apparatus) connected to the network N such as the Internet may provide at least one of the functions in the first processing circuitry 17, and the first memory 15 and the first communication interface 13. Here, the subject can use various functions of the clinical decision-making support apparatus 11 from the patient's own terminal apparatus 20 through the network N. FIG. 2 is a diagram illustrating one example of the clinical decision-making support system 1 that is achieved on the cloud. As illustrated in FIG. 2, the clinical decision-making support apparatus 11 is achieved by a server apparatus about the cloud. Here, the terminal apparatus 20 is formed by the user terminal 130 that can receive the input from the subject, and an in-hospital terminal 131 that can receive the input from the medical workers including the doctor.

The first communication interface 13 performs data communication with the external storage apparatus 3, the medical information storage apparatus 5, and the terminal apparatus 20, for example. The communication standard of the first communication interface 13 may be any standard. The first communication interface 13 outputs the data acquired by the data communication with the external storage apparatus 3, the medical information storage apparatus 5, and the terminal apparatus 20 to the first memory 15. The first communication interface 13 receives an electric signal of the input operation that is input in an input interface 29 of the terminal apparatus 20 through a second communication interface 21. The first communication interface 13 outputs the received electric signal to the first processing circuitry 17. The first communication interface 13 transmits to the terminal apparatus 20, various kinds of display data generated by the first processing circuitry 17, the response to the inquiry sent from the terminal apparatus 20, and the like. The display data generated by the first processing circuitry 17 is described below. The first communication interface 13 corresponds to a communication unit.

The first memory 15 is a storage apparatus such as a hard disk drive (HDD), a solid state drive (SSD), or integrated circuitry storage device storing various kinds of information. The first memory 15 may be, as an alternative to the HDD, the SSD, and the like, a driving device that reads and writes various pieces of information between the first memory 15 and a portable storage medium such as compact disc (CD), a digital versatile disc (DVD), or a flash memory or a semiconductor memory element such as a random access memory (RAM), for example.

The first memory 15 stores various computer programs about a system controlling function 171, an acquiring function 173, an analyzing function 174, a specifying function 175, a deciding function 177, and a calculating function 179. The first memory 15 stores the data acquired by the execution of the acquiring function 173, which is described below. For example, the first memory 15 stores the disease information, the insurance information, and the medical information acquired from the external storage apparatus 3. In addition, the first memory 15 stores the patient information, the personal information, and the language processing result of the subject that the acquiring function 173 acquires from the medical information storage apparatus 5. The disease information forms a database of the treatment methods for the disease (hereinafter referred to as treatment database) in the first memory 15, for example. Note that the first memory 15 may store the personal information acquired from the terminal apparatus 20. The first memory 15 may store therein the result of the medical practice by the treatment of another subject (hereinafter referred to as outcome). The first memory 15 stores various kinds of data generated by the execution of the analyzing function 174, the specifying function 175, the deciding function 177, and the calculating function 179. The system controlling function 171, the acquiring function 173, the analyzing function 174, the specifying function 175, the deciding function 177, and the calculating function 179 are described below. The first memory 15 corresponds to a storage unit.

The first processing circuitry 17 includes, for example, a processor such as a central processing unit (CPU), a micro processing unit (MPU), or a graphics processing unit (GPU), and a memory such as a read only memory (ROM) or a RAM as the hardware resource. The first processing circuitry 17 may be formed by application specific integrated circuitry (ASIC), a field programmable gate array (FPGA), a complex programmable logic device (CPLD), a simple programmable logic device (SPLD), or the like.

The first processing circuitry 17 includes the system controlling function 171, the acquiring function 173, the analyzing function 174, the specifying function 175, the deciding function 177, and the calculating function 179. The system controlling function 171, the acquiring function 173, the analyzing function 174, the specifying function 175, the deciding function 177, and the calculating function 179 are stored in the first memory 15 in a computer-executable format. The first processing circuitry 17 causes the processor, which executes the computer program developed in the memory, to perform the system controlling function 171, the acquiring function 173, the analyzing function 174, the specifying function 175, the deciding function 177, and the calculating function 179.

That is to say, the first processing circuitry 17 corresponds to the processor that reads out the computer program from the first memory 15 and executes the computer program so as to achieve the function corresponding to each computer program. In other words, the first processing circuitry 17 having read out each computer program has the function corresponding to the read computer program. Note that each function (171, 173, 174, 175, 177, 179) is not limited to the case of being achieved by single processing circuitry. A plurality of independent processors may be combined to form the processing circuitry and each processor may execute the computer program to achieve each function (171, 173, 174, 175, 177, 179). The procedure of the process of the acquiring function 173, the analyzing function 174, the specifying function 175, the deciding function 177, and the calculating function 179 that is achieved by the first processing circuitry 17 is described below in accordance with the procedure of performing the process (hereinafter referred to as decision-making support process) for supporting the decision of the option for the treatment method (hereinafter referred to as treatment option) for the disease of the subject.

The system controlling function 171 controls each function of the first processing circuitry 17 on the basis of the electric signal corresponding to the input operation received from the operator through the input interface 29. Specifically, the first processing circuitry 17 reads out the control computer program stored in the first memory 15, develops the control computer program on the memory in the first processing circuitry 17, and controls each part of the clinical decision-making support apparatus 11 in accordance with developed control computer program. The first processing circuitry 17 that performs the system controlling function 171 corresponds to a system control unit.

The acquiring function 173 acquires the disease information about the disease of the subject, the patient information of the subject, the medical information about the disease, and the personal information of the subject. Specifically, the acquiring function 173 acquires the disease information, the insurance information, and the medical information from the public database 31 in the external storage apparatus 3. In addition, the acquiring function 173 acquires the disease information from the external storage apparatus 3 in accordance with the update of the disease guideline. Here, the acquiring function 173 updates the database about the treatment method for the disease in accordance with the update of the disease guideline. Additionally, the acquiring function 173 may acquire the treatment method input through the terminal apparatus 20.

The acquiring function 173 acquires a search instruction (data such as audio and text) about the display content displayed on a display 27 from the terminal apparatus 20. The acquiring function 173 acquires the audio data of the subject input to the terminal apparatus 20 in a dialogic manner about the display content displayed on the display 27 from the terminal apparatus 20. The first processing circuitry 17 that performs the acquiring function 173 corresponds to an acquisition unit.

The analyzing function 174 analyzes the search instruction and the audio data. This analysis is, for example, natural language processing. The analyzing function 174 stores the language processing result corresponding to the result of analyzing the audio data or the search history of the search instruction in the first memory 15. Note that the analyzing function 174 may transmit the language processing result through the first communication interface 13 to the medical information storage apparatus 5 and stores the result therein. The process to be performed by the analyzing function 174 may be performed by the terminal apparatus 20 or the external server apparatus. In this case, the analyzing function 174 is unnecessary in the first processing circuitry 17.

The specifying function 175 specifies the options about the treatment method for the disease of the subject on the basis of the disease information, the patient information, and the medical information. For example, first, by using the disease name of the subject in the patient information, the specifying function 175 specifies the disease guideline in the disease information and the medical information about the disease name. Next, the specifying function 175 specifies the treatment method for the disease name as the treatment options about the treatment of the disease in accordance with a rule-based computer program, for example, by using the specified disease guideline and medical information. Note that the specifying function 175 may specify the treatment options by using a learned model that has learned so that the treatment options are output based on the input of the disease name of the subject, the guideline information, and the personal information such as the language processing result. Note that the number of specified treatment options may be either one or more than one. When the treatment method is input by the doctor's or subject's instruction through the terminal apparatus 20, the specifying function 175 specifies the input treatment method as the treatment option. Note that the specifying function 175 may specify the treatment options by additionally using the outcome of another subject.

Note that the specified treatment options may be correlated with, for example, the information expressing why this option is presented to the doctor or the subject (for example, medical information), the reliability of the specified treatment method, the five-year survival rate for the specified treatment method, and the ratio of patients with the same condition as the subject who has chosen this option. In addition, when the treatment method is different depending on the coverage of the insurance of the subject such as in the United States, the specifying function 175 may specify the treatment options within the coverage of the insurance of the subject.

The specified treatment options do not have to be continuous and may have some branches. For example, in the case where whether to perform the pharmacotherapy is determined based on the pathological examination of the specimen obtained in the surgery, there are branch time points to determine the treatment method by the subject and the doctor in the treatment option (hereinafter this time point is referred to as determination branch point). Therefore, the specified treatment option includes the treatment plan in the case of performing the pharmacotherapy after the determination branch point, and the treatment plan in the case of not performing the pharmacotherapy after the determination branch point. The determination branch point corresponds to the milestone about the choice of the treatment method for the subject. The first processing circuitry 17 that performs the specifying function 175 corresponds to a specifying unit.

The deciding function 177 decides the schedule of the treatment (hereinafter referred to as treatment schedule) about the treatment method for the disease of the subject on the basis of the disease information, the patient information, and the medical information in accordance with the specified treatment option. That is to say, as the treatment schedule, the deciding function 177 estimates the rough period of time required for the treatment of the disease on the basis of patient information that is the data about the subject (if the patient information is not stored in the first memory 15, general data about the disease in the treatment database) and the guideline information for the specified treatment option. Note that when the determination branch point exists in the treatment option, the deciding function 177 decides the treatment schedule in each treatment method after the determination branch point.

When the treatment option specified by the specifying function 175 is changed to another treatment option by the doctor's or subject's instruction through the terminal apparatus 20, for example, the deciding function 177 decides another treatment schedule about another treatment method for the new treatment option on the basis of the disease information, the patient information, and the medical information. Note that the deciding function 177 may predict and decide the event about the subject, that is, the priority item using the language processing result corresponding to the analysis result by the analyzing function 174. The priority item is predicted by, for example, inputting the language processing result to the learned model that outputs the priority item on the basis of the input of the language processing result. The first processing circuitry 17 that performs the deciding function 177 corresponds to a decision unit.

The calculating function 179 calculates the event schedule about the event specific to the subject in the period for which the treatment method in the specified treatment option is to be performed, on the basis of the personal information, the treatment method, and the treatment schedule. The event specific to the subject corresponds to the aforementioned priority item, for example. Note that when the determination branch point exists in the treatment option, the calculating function 179 decides the treatment method after the determination branch point and the event schedule in the period for which the treatment method is to be performed. The calculating function 179 calculates another event schedule about the event specific to the subject in the period for which another treatment is to be performed, on the basis of the personal information, another treatment method, and another treatment schedule. In this case, the display data (display content) that expresses the other event schedule and the event about the other treatment schedule and the other treatment schedule together with the other options in time series is transmitted to the terminal apparatus 20 by the first communication interface 13. The first processing circuitry 17 that performs the calculating function 179 corresponds to a calculation unit. One example of the process to be performed by the calculating function 179 is described below.

For example, in a case where the subject inputs the personal information with the priority item “want a child in the future” through the terminal apparatus 20, the calculating function 179 calculates the period until the start of the chemotherapy for each of the treatment options specified by the specifying function 175 as the event schedule about the fertility of the subject. If the personal information includes the basal body temperature of the subject, the calculating function 179 predicts (calculates) the ovulation day of the subject as the event specific to the subject on the basis of the change of the basal body temperature in the event schedule about the fertility.

Note that the calculating function 179 may calculate the event schedule by using the medical information additionally. For example, in the case where the subject inputs the personal information with the priority item “expense” through the terminal apparatus 20, the calculating function 179 calculates the approximate value of the expense for the treatment method (hereinafter referred to as treatment expense approximate value) by using the treatment insurance point according to the treatment method in the treatment option in accordance with the treatment schedule. Additionally, the calculating function 179 calculates the necessary amount of drug used in the treatment method by further using the weight and height of the subject in the patient information. Next, the calculating function 179 calculates the expense for the drug used in the treatment method in accordance with the treatment schedule by applying the calculated necessary amount and the price of the drug to the existing drug cost calculation application. The drug cost calculation application may be stored in the first memory 15, the external storage apparatus 3, the medical information storage apparatus 5, or the like.

In a case where the subject inputs the personal information with the priority item “expense” through the terminal apparatus 20, the calculating function 179 may calculate the upper limit of the patient's burden in the treatment method in the treatment option on the basis of the monthly remuneration and the standard monthly remuneration of the subject or the subject's household. Here, the calculating function 179 calculates the information about the expense including the upper limit of the patient's burden in addition to the treatment expense approximate value as the monthly event schedule in accordance with the treatment schedule. Moreover, by additionally using the information about the content of the private cancer insurance of the subject, the benefit from the municipality, and the like, the calculating function 179 may calculate the amount of benefit paid to the subject or the date when the benefit will be paid in the treatment schedule as the event schedule including the event specific to the subject.

For example, in a case where the subject inputs the personal information with the priority item “hair loss” or “class visit” through the terminal apparatus 20, the calculating function 179 acquires the drug with the strong side effect of losing hair from the treatment database in each treatment option specified by the specifying function 175, and calculates the time when the hair loss begins and how long the hair loss continues as the general data. Next, the calculating function 179 acquires the date of the class visit as the event specific to the patient from the family schedule in the personal information of the subject, and generates the event schedule using the hair loss period and the date of the class visit.

The terminal apparatus 20 includes the second communication interface 21, a second memory 23, second processing circuitry 25, the display 27, and the input interface 29. The terminal apparatus 20 is not limited to a personal computer or a mobile terminal the subject borrows from the hospital and may be wireless hardware such as a personal computer, a tablet terminal, or a smartphone the subject or the like owns. Here, the terminal apparatus 20 is connected wirelessly to the network N through the second communication interface 21. The terminal apparatus 20 is operated by the doctor or the subject. To make the description specific, the terminal apparatus 20 is described below as a mobile terminal capable of wireless communication.

The second communication interface 21 performs data communication with the first communication interface 13 through the network N. The second communication interface 21 may perform the data communication with the external storage apparatus 3 or the medical information storage apparatus 5 through the network N. The second communication interface 21 transmits the electric signal of the input operation input to the input interface 29 to the clinical decision-making support apparatus 11.

For example, the second communication interface 21 transmits the personal information including the priority item and the subject's instruction input through the input interface 29 to the first communication interface 13. The second communication interface 21 receives the data (hereinafter referred to as display data) with the display content about the treatment option from the first communication interface 13 through the network N. The display data is the data expressing the event and the event schedule related to the treatment schedule, and the treatment schedule for each option in time series. The second communication interface 21 stores the received display data in the second memory 23.

The second memory 23 is a storage apparatus such as an HDD, an SSD, or an integrated circuitry storage device that stores various kinds of information. The second memory 23 may be a driving device that reads or writes various pieces of information between the second memory 23 and a portable storage medium such as a CD, a DVD, or a flash memory, a semiconductor memory element such as a RAM, or the like in addition to the HDD and the SSD, for example. The second memory 23 stores the information transmitted from the first communication interface 13, for example the display data, through the second communication interface 21. The second memory 23 stores the computer program about a displaying function 251.

The second processing circuitry 25 includes, for example, a processor such as a CPU, an MPU, or GPU, and a memory such as a ROM or a RAM as the hardware resource. The second processing circuitry 25 may be formed by an ASIC, an FPGA, a CPLD, an SPLD, or the like. The second processing circuitry 25 includes the displaying function 251 and the like. The displaying function 251 is stored in the second memory 23 as a computer-executable computer program. The second processing circuitry 25 causes a processor, which executes the computer program developed in the memory, to perform the displaying function 251. That is to say, the second processing circuitry 25 corresponds to the processor that reads out the computer program from the second memory 23 and executes the computer program so as to achieve the function corresponding to the computer program.

In other words, the second processing circuitry 25 having read the computer program has the function corresponding to the read computer program. Note that the displaying function 251 is not limited to the displaying function achieved by single processing circuitry. A plurality of independent processors may be combined to form the processing circuitry and each processor may execute the computer program so as to achieve the displaying function 251. The second processing circuitry 25 for performing the displaying function 251 is one example of a display controlling unit. The second processing circuitry 25 causes the displaying function 251 to control the display 27 in various displays on the display 27. Thus, the displaying function 251 causes the display 27 to display the display content.

The display 27 displays various kinds of information. For example, the display 27 outputs the graphical user interface (GUI) for receiving various operations from the operator. As the display 27, for example, a liquid crystal display (LCD), a cathode ray tube (CRT) display, an organic electroluminescence display (OELD), a plasma display, or any other display can be used as appropriate. Note that the display 27 may be formed as a touch screen in which a display and a touch pad (input interface 29) are unified.

The displaying function 251 reads out various kinds of information, which is sent from the clinical decision-making support apparatus 11 through the first communication interface 13, from the second memory 23, and the display 27 displays the information. For example, the display 27 displays the event schedule and the event related to the treatment schedule, and the treatment schedule for each treatment option in time series. The display 27 displays the questions necessary to analyze (natural language processing) the priority item, for example, about the private insurance. The specific display content that is displayed on the display 27 is described below in accordance with the procedure to perform the decision-making support process. The display 27 corresponds to a display unit.

The input interface 29 receives various input operations from the operator, for example the subject, converts the received input operation into the electric signal, and outputs the electric signal to the second processing circuitry 25. The converted electric signal is output to the first processing circuitry 17 through the second communication interface 21 and the first communication interface 13. For example, the input interface 29 receives the choosing instruction, various conditions, and the like from the operator. As the input interface 29, for example, a mouse, a keyboard, a track ball, a switch, a button, a joystick, a touch pad, a touch panel display, or the like can be used as appropriate.

In the present embodiment, the input interface 29 is not limited to the input interface including a physical operation component such as a mouse, a keyboard, a track ball, a switch, a button, a joystick, a touch pad, or a touch panel display. For example, processing circuitry that receives the electric signal corresponding to the input operation from the external input apparatus provided separately from the device and outputs this electric signal to the first processing circuitry 17 and the second processing circuitry 25 is also included in the examples of the input interface 29. In addition, the input interface 29 may be formed by a tablet terminal or the like that can wirelessly communicate with the clinical decision-making support apparatus 11.

The input interface 29 inputs the personal information of the subject by the subject's instruction. For example, the input interface 29 inputs the instruction to change the priority item or the presented options to the conditions that the subject prefers (hereinafter this instruction is referred to as changing instruction), or inputs the search instruction about the display content regarding the display data. The changing instruction corresponds to the instruction for changing the specified treatment option to another treatment option. Note that the doctor or the subject may input the treatment option with the input interface 29. Moreover, the input interface 29 inputs the answer to the question necessary for the natural language processing about the priority item in text, for example. Note that the input interface 29 may input the doctor's or subject's voice in a dialogic manner about the display content regarding the display data. The input interface 29 corresponds to an input unit.

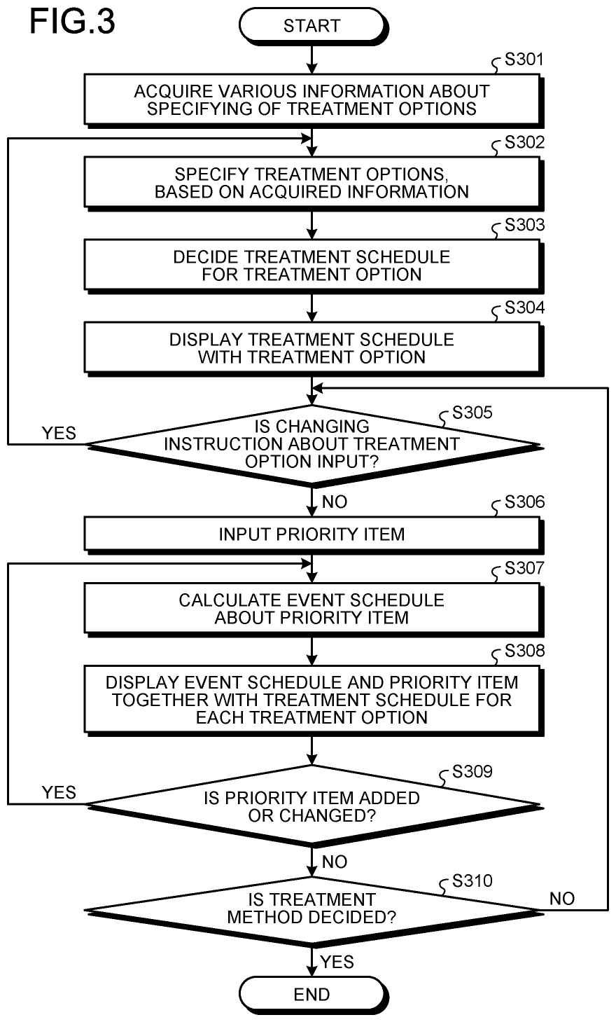

The entire structure of the clinical decision-making support system 1 has been described. One example of the procedure for performing the decision-making support process is described below. To make the description specific, the breast cancer is taken as the example of the disease of the subject regarding the decision-making support process. Note that the disease of the subject regarding the decision-making support process is not limited to the breast cancer and may be any other disease. FIG. 3 is a flowchart illustrating one example of the procedure of the decision-making support process. Note that the decision-making support process may be performed not just before the start of the treatment for the disease of the subject but also in the middle of the treatment for the disease of the subject.

Decision-Making Support Process

Step S301

The acquiring function 173 acquires various kinds of information about the specifying of the treatment options (for example, disease information, patient information, medical information, and personal information). Note that the acquiring function 173 may acquire the treatment method input from the terminal apparatus 20. The information acquired by the acquiring function 173 is stored in the first memory 15.

Step S302

The specifying function 175 specifies the treatment options on the basis of the acquired information. The treatment options are specified using the rule-based computer programs or the learned model, for example. In order to make the description specific, it is assumed that there are two options, an option A and an option B, as the treatment options. Each of the option A and the option B includes two determination branch points.

Step S303

The deciding function 177 decides the treatment schedule on the basis of the disease information, the patient information, and the medical information in each of the treatment options. When the treatment option includes the determination branch point, the deciding function 177 decides the treatment schedule about the treatment method before the determination branch point and the treatment schedule about the treatment methods after the determination branch point. After this step, the first communication interface 13 transmits the treatment schedule and the like to the second communication interface 21 together with the treatment option. The second communication interface 21 causes the second memory 23 to store therein the received treatment option and treatment schedule, for example.

Step S304

The treatment schedule is displayed with the treatment option. Specifically, the displaying function 251 causes the display 27 to display the treatment option and the treatment schedule and the like stored in the second memory 23. Thus, the chronological treatment schedule for each treatment option is presented to the subject through the display 27 of the terminal apparatus 20. Note that the displaying function 251 may cause the display 27 to further display the standard treatment schedule about the disease, that is, the treatment schedule about the standard treatment on the basis of the disease information about the disease of the subject as the reference information. In this case, the displaying function 251 may cause the display 27 to additionally display the name of the doctor in charge (or attending physician) about the disease of the subject, for example. The name of the doctor in charge is added to the chronological treatment schedule for each treatment option.

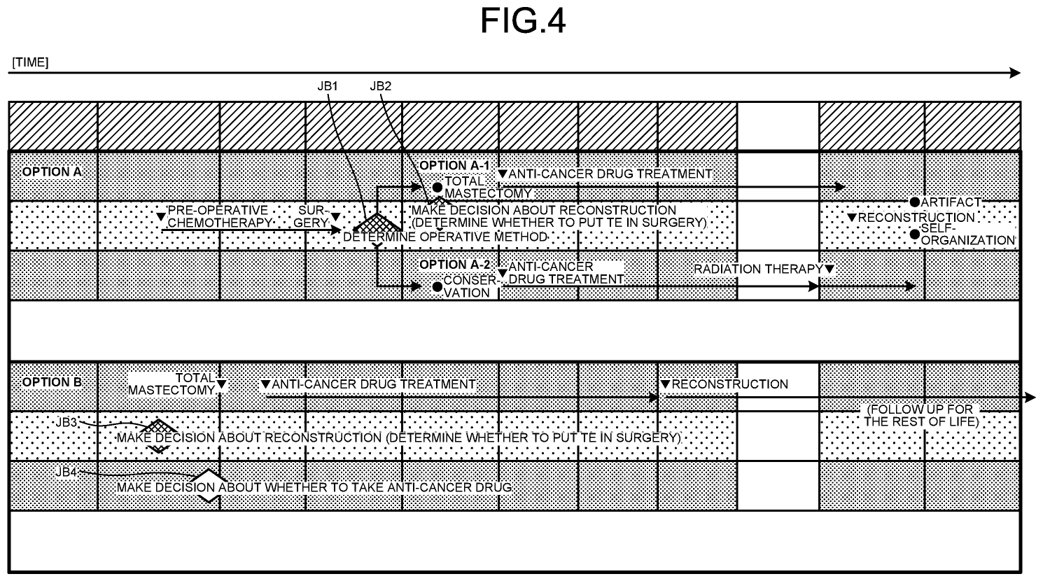

FIG. 4 is a diagram illustrating one example of the treatment schedule for the option A and the option B. The display data illustrated in FIG. 4 is displayed on the display 27 of the terminal apparatus 20. The order of arranging the option A and the option B in FIG. 4 can be set optionally. The options can be rearranged as appropriate in accordance with the subject's or doctor's instruction through the input interface 29, for example in the order of the five-year survival rate. The options may be arranged in the order that is set in advance.

The treatment method of the option A in FIG. 4 is the pre-operative chemotherapy. In determining the operative method at a first determination branch point JB1 for the option A, whether the breast is conserved or removed entirely can be chosen. At the first determination branch point JB1, the treatment method of an option A-1 indicates the total mastectomy. In the total mastectomy, the determination about the reconstruction is presented at a second determination branch point JB2. The determination about the reconstruction corresponds to the determination as to whether a tissue expander (TE) is set in the surgery of the total mastectomy. The treatment schedule for the option A-1 includes the anti-cancer drug treatment and the reconstruction using the artifact or the self-organization.

In a case of the treatment method of setting the artifact or using the self-organization in the treatment method including the breast reconstruction performed after the second determination branch point JB2 in FIG. 4, the treatment schedule that is decided for such a treatment method may be displayed on the display 27 chronologically. At the first determination branch point JB1, the treatment method for an option A-2 indicates the breast conservation. The treatment schedule for the option A-2 includes the anti-cancer drug treatment and the radiation therapy.

The treatment method for the option B in FIG. 4 indicates the total mastectomy. The determination about the reconstruction is presented at a third determination branch point JB3 for the option B. At a fourth determination branch point JB4 for the option B, the determination about whether to use the anti-cancer drug is presented. The treatment schedule for the option B includes the anti-cancer drug treatment and the reconstruction.

When the operator's instruction through the input interface 29 has chosen one of the options (option A, option A-1, option A-2, and option B) or put a cursor on one option, for example, the display 27 may display the medical information to explain the reason for the treatment option, the reliability, the five-year survival rate, the ratio of patients with the same condition as the subject who have chosen the option, and the like in a pull-down form or pop-up form. Instead of choosing the treatment option, the option of “not treating” may be selected by the instruction of the operator.

Step S305

If the changing instruction about the condition of the treatment option is input through the input interface 29 (Yes at step S305), the process from step S302 to step S304 is repeated under the changed condition for the treatment option. If the changing instruction about the condition of the treatment option is not input through the input interface 29 (No at step S305), the process at step S306 is performed. The specified treatment option may be changed to another treatment option by the subject's or doctor's instruction through the terminal apparatus 20 in this step (Yes at step S305). Here, for the other treatment option, the processes at step S303 and step S304 are repeated.

Step S306

The priority item is input through the input interface 29. Note that in the case where the personal information acquired by the acquiring function 173 includes the priority item or the priority item is already set, the priority item can be changed, reset, or input as appropriate in this step. The input of the priority item may be the output result based on the input of the language processing result into the learned model that outputs the priority item on the basis of the input of the doctor, the subject, and the language processing result. This step may come before step S304.

Step S307

The calculating function 179 calculates the event schedule about the priority item. The event schedule is calculated in accordance with each treatment option. The calculated event schedule is correlated with the treatment option. In the case where the treatment option includes the determination branch point, the calculating function 179 calculates the event schedule before the determination branch point and the event schedule after the determination branch point. The first communication interface 13 correlates the signed event schedule and the treatment schedule with the treatment option and transmits the schedules and the treatment option to the second communication interface 21.

Step S308

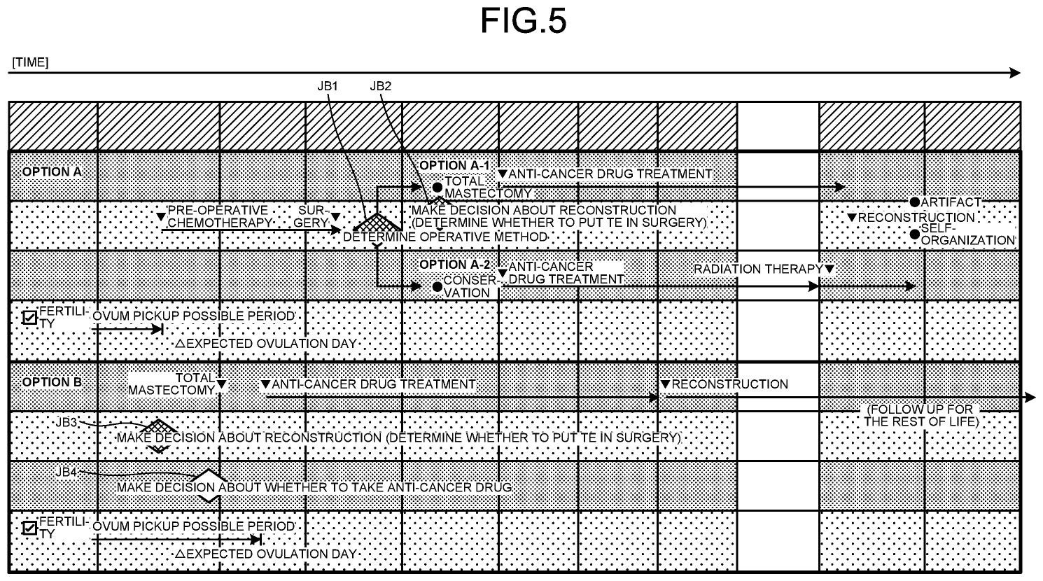

The event schedule and the priority item related to the treatment schedule are displayed chronologically for each treatment option with the treatment schedule. Note that the event schedule and the priority item related to the treatment schedule may be displayed additionally on the display 27 as the reference information together with the standard treatment schedule about the disease of the subject. In addition, the display 27 may display the name of the doctor in charge (or attending physician) about the disease of the subject, for example. FIG. 5 is a diagram illustrating one example of the display content displayed on the display 27 about the display data transmitted from the clinical decision-making support apparatus 11 to the terminal apparatus 20. FIG. 5 illustrates the ovum pickup possible period as the event schedule in the case where the priority item is fertility. If the basal body temperature is included in the personal information, the scheduled ovum pickup date is presented as the event specific to the subject as illustrated in FIG. 5 on the basis of the ovulation day predicted by the calculating function 179. Note that the order of arranging the option A and the option B in FIG. 5 can be set optionally, and for example, the option A and the option B may be set in the order of satisfying the priority, that is, in the order of the longer revisit possible period.

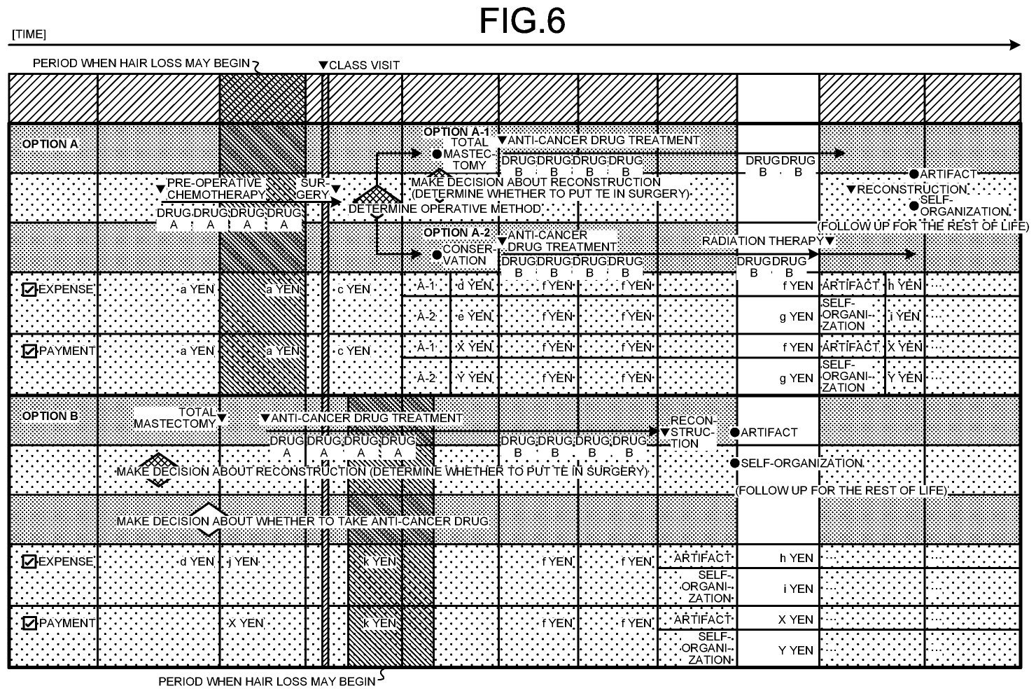

FIG. 6 is a diagram illustrating one example of the display content displayed on the display 27 about the display data transmitted from the clinical decision-making support apparatus 11 to the terminal apparatus 20. FIG. 6 illustrates the event schedule displayed on the display 27 in the case where the priority items are expense, payment, hair loss, and class visit. As illustrated in FIG. 6, the expense and the payment timing for the drugs and the procedure of the treatment method that are used in the treatment schedule, the amount and payment timing of the insurance claims and benefits to be paid, the class visit date, and the hair loss period due to the treatment are displayed chronologically as the event schedule for each treatment option along with the treatment schedule.

In addition, as illustrated in FIG. 6, the display 27 displays chronologically the event schedule for the events including the expense and the payment for each treatment method in the case where the artifact is put into the subject or in the case where the self-organization is used in the treatment method of the breast reconstruction. As illustrated in FIG. 6, the drug that may result in the hair loss is displayed with emphasis for each of the option A and the option B. The hair loss and the class visit in FIG. 6 may be written below the priority item “payment”.

In addition, in the case where there are a plurality of priority items as illustrated in FIG. 6, the index (weighting) expressing the priority item based on which the treatment option is specified may be displayed for each option. For example, in FIG. 6, four priority items (expense, payment, hair loss, and class visit) are displayed for each of the two treatment options (option A, option B); however, in each of the option A (including option A-1 and option A-2) and the option B, the proportion of the priority in the four priority items, that is, the degree of the importance of the priority of the event may be displayed in a pie chart. The proportion of the priority is calculated by the calculating function 179 at step S307, for example. The proportion of the priority is calculated based on the ratio of overlap between the treatment schedule and the event schedule of each priority item, for example.

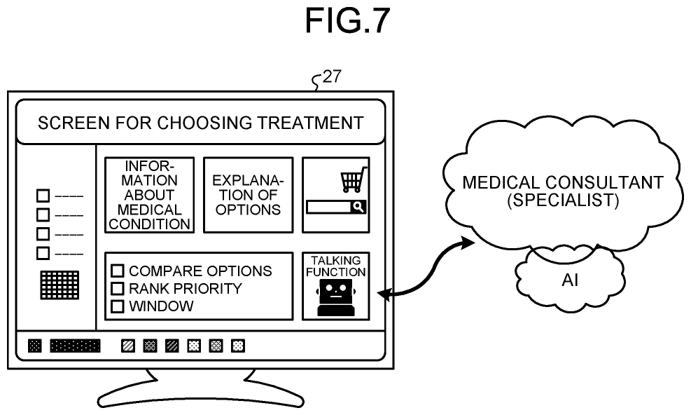

FIG. 7 is a diagram illustrating another example of the display content displayed on the display 27 about the display data transmitted from the clinical decision-making support apparatus 11 to the terminal apparatus 20. As illustrated in FIG. 7, the display screen of the display 27 may include, in addition to the display content displaying the event schedule and the priority items together with the chronological treatment schedule for each treatment option, the screen displaying information about the subject's condition, the treatment options, a search window for various kinds of information, and the screen for the subject to talk with the specialist, AI, or family, for example.

Step S309

If the addition or the change of the priority item is input through the input interface 29 (Yes at step S309), the process in step S307 and S308 is repeated. If the addition or the change of the priority item is not input through the input interface 29 (No at step S309), the process in step S310 is performed.

Step S310

If SDM is carried out about the treatment method between the doctor and the subject (Yes at step S310), the decision-making support process ends. The decision of Yes at step S310 is made based on the mutual agreement between the subject and the doctor. The subject may also individually input Yes at step S310 and end the decision-making support process. Here, the first memory 15 stores the treatment option specified by the specifying function 175, the treatment schedule, the event schedule, and the event as the database (hereinafter referred to as co-operation decision database) therein in a manner correlating these pieces of information with each other. Note that the outcome of the subject may also be correlated and stored in the co-operation decision database. The co-operation decision database is used in the process at step S302 and step S303 in the decision-making support process about other subjects with the disease similar to that of the subject. If SDM is not carried out (No at step S310), the process at step S305 is repeated to change various things about the treatment option and change the priority item.

The clinical decision-making support apparatus 11 according to the embodiment described above acquires the disease information, the patient information, the medical information, and the personal information, specifies the options about the treatment method for the disease of the subject, based on the disease information, the patient information, and the medical information, decides the treatment schedule about the treatment method for the option, based on the disease information, the patient information, and the medical information, calculates the event schedule about the event specific to the subject in the period for which the treatment method for the option is to be performed, based on the personal information, the treatment method, and the treatment schedule, and transmits to the terminal apparatus 20, the display data with the display content expressing the treatment schedule, the event schedule, and the event for each option in time series.

Thus, by the use of the present clinical decision-making support apparatus 11, the event unique to the subject can be displayed on the display 27 in accordance with the treatment option together with the treatment schedule of the treatment method and the event schedule in time series. Therefore, the present clinical decision-making support apparatus 11 makes it possible to see the treatment schedule of the treatment method and the event schedule chronologically. Thus, the subject can understand, in a course of treatment, a variety of factors including the medical aspects of the treatment methods, the money, the family schedules, the work, and such personal information as whether to have children in the future, which are the basis for making decisions on the treatment method, from a wider view point for each treatment method. Accordingly, the present clinical decision-making support apparatus 11 allows the subject to choose the treatment method with less regret; therefore, the efficiency of SDM between the doctor and the subject can be improved and the subject satisfaction in SDM can be improved.

First Modification

In this modification, the decision-making support process is performed not just before or at the start of the treatment for the disease of the subject but also in the middle of the treatment as appropriate. In this case, at step S301, the acquiring function 173 acquires the information about the treatment that is already done to the subject and the current treatment status from the patient information, for example. Next, at step S302, the specifying function 175 specifies the treatment option by additionally using the information about the completed treatment and the treatment status. Thus, in the present modification, the treatment method can be reviewed at any stage of the treatment on the basis of the treatment status or the event unique to the subject at the current time point, so that the efficiency of SDM and the satisfaction of the subject in SDM can be improved.

Second Modification

In this modification, at step S302 in the decision-making support process, the specified treatment options are compared about at least one of benefit/risks, advantages/disadvantages, and pros/cons and displayed on the display 27 of the terminal apparatus 20. In order to make the description specific, it is assumed that the treatment options specified by the specifying function 175 are three kinds: option A, option D, and option C.

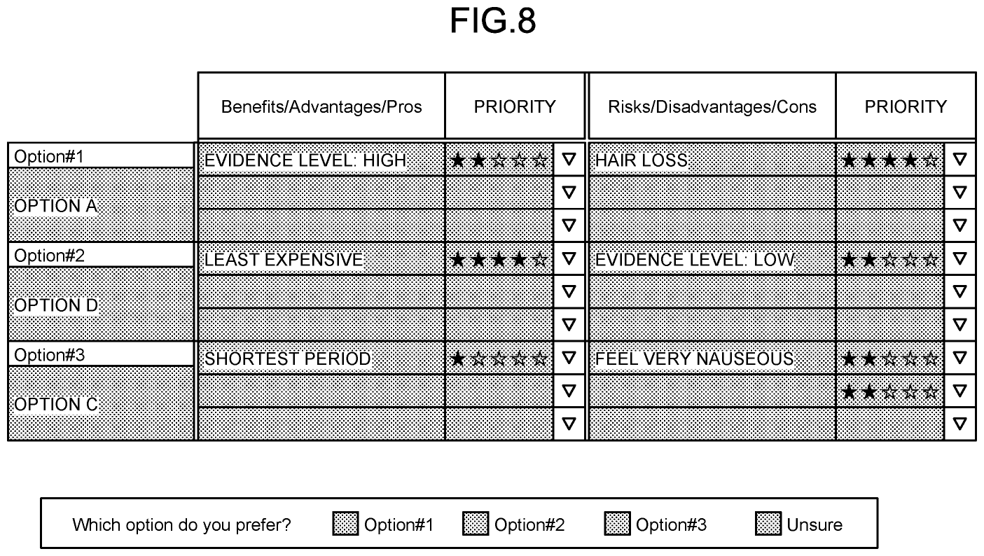

FIG. 8 is a diagram illustrating an example in which the three kinds of treatment options, option A, option D, and option C, are compared about at least one of benefit/risks, advantages/disadvantages, and pros/cons, and the priority input by the subject is displayed. The priority corresponds to the index expressing the degree of priority for the subject about benefit/risks, advantages/disadvantages, and pros/cons in choosing the treatment method, for example.

At step S302, the comparison of benefit/risks, advantages/disadvantages, and pros/cons for each option is displayed on the display 27 in the format in which the subject can input the priority. On this display, the subject inputs the priority through the input interface 29. The acquiring function 173 acquires the input priority through the first and second communication interfaces and the network N.

At step S304, the treatment schedule is displayed with the treatment option in the order of higher priority in the treatment options. At step S307, the first communication interface 13 transmits the display data with the display content in which the treatment options are arranged in accordance with the priority to the terminal apparatus 20. At step S308, the event schedule and the priority item are displayed chronologically and in the order from the highest priority in the treatment options, along with the treatment schedule for each treatment option.

In the clinical decision-making support apparatus 11 according to this modification, the terminal apparatus 20 displays the specified treatment options after comparing at least one of benefit/risks, advantages/disadvantages, and pros/cons, and inputs the priority about the treatment option for each treatment option, the priority is further acquired from the terminal apparatus 20, and the display data with the display content in which the treatment options are arranged in accordance with the priority is transmitted to the terminal apparatus 20. Thus, by the present clinical decision-making support apparatus 11, the treatment options can be displayed on the display 27 with the event schedule and the treatment schedule in the order of the priority desired by the subject. Therefore, by the present clinical decision-making support apparatus 11, the efficiency of SDM and the satisfaction of the subject in SDM can be improved further.

In the case of implementing the technical concept of the present embodiment with the clinical decision-making support system 1, the clinical decision-making support system 1 includes the terminal apparatus 20 including the input unit with which the subject inputs the personal information, the acquisition unit that acquires the disease information about the disease of the subject, the patient information of the subject, the medical information about the disease, and the personal information of the subject, the specifying unit that specifies the options about the treatment method for the disease of the subject, based on the disease information, the patient information, and the medical information, the decision unit that decides the treatment schedule about the treatment method for the specified option, based on the disease information, the patient information, and the medical information, the calculation unit that calculates the event schedule about the event specific to the subject in the period for which the treatment method for the option is to be performed, based on the personal information, the treatment method, and the treatment schedule, and the display unit mounted on the terminal apparatus 20 for displaying the display content expressing the treatment schedule, the event schedule, and the event for each option in time series. Since the procedure and the effect of the decision-making support process to be performed by the clinical decision-making support system 1 are similar to those of the embodiment, the description is omitted.

In the case of implementing the technical concept of the present embodiment with the clinical decision-making support computer program, the clinical decision-making support computer program causes a computer to perform: acquiring the disease information about the disease of the subject, the patient information of the subject, the medical information about the disease of the subject, and the personal information about at least the event that is specific to the subject; specifying the options about the treatment method for the disease, based on the disease information, the patient information, and the medical information; deciding the treatment schedule about the treatment method for the option, based on the disease information, the patient information, and the medical information; calculating the event schedule about the specific event in the period for which the treatment method for the option is to be performed, based on the personal information, the treatment method, and the treatment schedule; and transmitting to the terminal apparatus 20, the display data expressing the event schedule and the event related to the treatment schedule, and the treatment schedule for each option in time series.

For example, the decision-making support process can be performed by installing the decision-making support computer program on a computer in HIS and developing this computer program on the memory. Here, the computer program that causes the computer to perform this method can be stored in a storage medium such as a magnetic disk (for example, hard disk), an optical disk (for example, CD-ROM or DVD), or a semiconductor memory and distributed. Since the procedure and the effect of the decision-making support computer program are similar to those of the embodiment, the description is omitted.

In the case of implementing the technical concept of the present embodiment with the clinical decision-making support method, the clinical decision-making support method includes: acquiring the disease information about the disease of the subject, the patient information of the subject, the medical information about the disease of the subject, and the personal information about at least the event that is specific to the subject; specifying the options about the treatment method for the disease, based on the disease information, the patient information, and the medical information; deciding the treatment schedule about the treatment method for the option, based on the disease information, the patient information, and the medical information; calculating the event schedule about the specific event in the period for which the treatment method for the option is to be performed, based on the personal information, the treatment method, and the treatment schedule; and transmitting to the terminal apparatus, the display data expressing the event schedule and the event related to the treatment schedule, and the treatment schedule for each option in time series. Since the procedure and the effect of the decision-making support method are similar to those of the embodiment, the description is omitted.

According to at least one embodiment described above, the clinical decision-making support apparatus 11, the clinical decision-making support system 1, and the clinical decision-making support computer program that can improve the efficiency of SDM between the doctor and the subject can be provided.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A clinical decision-making support apparatus comprising:
processing circuitry configured to
acquire disease information about a disease of a subject, patient information of the subject, medical information about the disease, and personal information about at least an event that is specific to the subject,
specify an option about a treatment method for the disease, based on the disease information, the patient information, and the medical information,
decide a treatment schedule about the treatment method for the option, based on the disease information, the patient information, and the medical information, and
calculate an event schedule about the specific event in a period for which the treatment method for the option is to be performed, based on the personal information, the treatment method, and the treatment schedule; and
a communication interface configured to transmit, to a terminal apparatus, display data expressing the event schedule and the event related to the treatment schedule, and the treatment schedule for each option in time series.

2. The clinical decision-making support apparatus according to claim 1, wherein
at least one of the processing circuitry and the communication interface is provided to a server apparatus in at least one of a network and a hospital, and
the terminal apparatus is operated by the subject.

3. The clinical decision-making support apparatus according to claim 1, wherein
the processing circuitry is configured to
decide another treatment schedule about another treatment method for another option, based on the disease information, the patient information, and the medical information, and
calculate another event schedule about the specific event in a period for which the other treatment method is to be performed, based on the personal information, the other treatment method, and the other treatment schedule, and
the communication interface is configured to transmit, to the terminal apparatus, display data expressing the other event schedule and the event related to the other treatment schedule, and the other treatment schedule with the other option in time series.

4. The clinical decision-making support apparatus according to claim 1, further comprising a memory configured to store the specified option, the treatment schedule, the event schedule, and the event as a database in a manner correlating the specified option, the treatment schedule, the event schedule, and the event with each other.

5. The clinical decision-making support apparatus according to claim 1, wherein
the terminal apparatus is configured to
display the option after comparing at least one of benefit/risk, advantage/disadvantage, and pros/cons, and
input priority about the option for each option,
the processing circuitry is configured to further acquire the priority from the terminal apparatus, and
the communication interface is configured to transmit, to the terminal apparatus, display data in which the option is arranged in accordance with the priority.

6. The clinical decision-making support apparatus according to claim 1, wherein
the terminal apparatus is configured to input a search instruction about display content regarding the display data, and
the processing circuitry is configured to
acquire the search instruction from the terminal apparatus,
analyze the search instruction,
specify the option by additionally using an analysis result of the search instruction, and
decide the event using the analysis result.

7. The clinical decision-making support apparatus according to claim 1, wherein
the terminal apparatus is configured to input voice of the subject in a dialogic manner about display content regarding the display data, and
the processing circuitry is configured to
acquire data of the voice from the terminal apparatus,
analyze the data of the voice,
specify the option by additionally using an analysis result of the data of the voice, and
decide the event, based on the analysis result of the data of the voice.

8. A clinical decision-making support system comprising:
a terminal apparatus including an input unit with which a subject inputs personal information about at least an event that is specific to the subject;
processing circuitry configured to
acquire disease information about a disease of the subject, patient information of the subject, medical information about the disease, and the personal information,
specify an option about a treatment method for the disease, based on the disease information, the patient information, and the medical information,
decide a treatment schedule about the treatment method for the option, based on the disease information, the patient information, and the medical information, and
calculate an event schedule about the specific event in a period for which the treatment method for the option is to be performed, based on the personal information, the treatment method, and the treatment schedule; and
a display configured to display the event schedule and the event related to the treatment schedule, and the treatment schedule for each option in time series.

9. A clinical decision-making support method comprising:
acquiring disease information about a disease of a subject, patient information of the subject, medical information about the disease, and personal information about at least an event that is specific to the subject;
specifying an option about a treatment method for the disease, based on the disease information, the patient information, and the medical information;
deciding a treatment schedule about the treatment method for the option, based on the disease information, the patient information, and the medical information;
calculating an event schedule about the specific event in a period for which the treatment method for the option is to be performed, based on the personal information, the treatment method, and the treatment schedule; and
transmitting, to the terminal apparatus, display data expressing the event schedule and the event related to the treatment schedule, and the treatment schedule for each option in time series.
